(19) United States
(12) Patent Application Publication
(10) Pub. No.: US 2024/0361291 A1
(43) Pub. Date: Oct. 31, 2024

(54) SOFT LEAF TISSUE ELASTICITY TESTER AND USE THEREOF FOR MEASURING PLANT HEALTH

(71) Applicants: The United States of America, as represented by the Secretary of Agriculture, Washington, DC (US); INTELLIGENT SENTINEL LLC, PHILADELPHIA, PA (US)

(72) Inventors: CHERYL M. ARMSTRONG, ABINGTON, PA (US); JOSEPH A. CAPOBIANCO, JR., MARKETING, NJ (US); YONG PING DUAN, FORT PIERCE, FL (US); WAN Y. SHIH, BRYN MAWR, PA (US); WEI HENG SHIH, BRYN MAWR, PA (US)

(21) Appl. No.: 18/645,684

(22) Filed: Apr. 25, 2024

Related U.S. Application Data
(60) Provisional application No. 63/498,972, filed on Apr. 28, 2023.

(51) Int. Cl. G01N 33/00; G01N 3/20

(52) U.S. Cl. CPC G01N 33/0098; G01N 3/20; G01N 2203/0023; G01N 2203/0051; G01N 2203/0075; G01N 2203/0623

(57) ABSTRACT

Described herein is a soft leaf tissue elasticity tester comprising a modified piezoelectric finger (PEF) sensor and a sample pad useful for rapidly and sensitively screening plants for disease by quantifying changes in tissue elasticity and/or stiffness as an indication of plant disease. Also described is a method of screening plants for disease by quantifying changes in tissue elasticity and/or stiffness as an indication of infection. The tissue elasticity measurements used to screen plants for disease may be performed using the soft tissue elasticity tester described.

### CROSS-REFERENCE TO RELATED APPLICATIONS

This application claims priority to U.S. Provisional Patent Application 63/498,972, filed Apr. 28, 2023, the contents of which is expressly incorporated herein by reference in their entirety.

### FIELD OF THE DISCLOSURE

The subject matter described herein is in the field of plant biology. Specifically, the subject matter relates to a detection device capable of quantifying soft leaf tissue elasticity, and a process for screening plants for disease rapidly and sensitively by using such device.

### SEQUENCE LISTING

The instant application contains a Sequence Listing XML required by 37 C.F.R. § 1.831 (a) which has been submitted in XML file format via the USPTO patent electronic filing system, and is hereby incorporated by reference in its entirety. The XML file was created on Apr. 25, 2024, is named Sequence_Listing-0039.22.xml, and has 3.30 KB.

### BACKGROUND OF THE DISCLOSURE

Although citrus is susceptible to multiple different pathogens, Candidatus Liberibacter asiaticus (Las) is currently the pathogen of greatest concern in the United States. Las, an alpha-proteobacterium, has been associated with the disease known as Huanglongbing (HLB or citrus greening). The disease has been found to affect all members of the genus Citrus and is systemic in the plant. Therefore, symptoms can be found on the leaves, fruit, and roots. Symptoms include leaf mottling, yellow shoots, and the production of small lopsided fruit with a bitter taste and aborted seeds. Twig/branch dieback, increased early fruit drop, and death of the tree are all markers of advanced disease states. The disease is primarily spread by transfer of the bacterium via feeding by Diaphorina citri Kuwayama (Asian citrus psyllid), although transmission via grafting and dodder (Cuscuta pentagona) is also possible.

Current control efforts in locations where HLB is already widespread include the use of insecticide programs to reduce the psyllid population and the application of additional nutrients/amendments to help extend the production life of HLB-affected citrus trees. In locations where the presence of HLB is sparse, such as the state of California, USA roguing of infected trees is also being used to help prevent spread. Nevertheless, complete eradication in California has proven difficult over the years because of the possibility of multiple introductions of the disease to the area, the consistent presence of the psyllid in the citrus-producing areas, and the long latency period of the disease leading to difficulties in identifying infected trees. Broad surveys are being conducted in citrus-producing regions of California entailing the testing of both psyllids and plants via real-time polymerase chain reaction (RT-PCR) as a proactive measure for identifying the presence of Las in a timely fashion. However, issues associated with disease detection, such as the uneven distribution of the pathogen in the plant, the reliance upon a lab-based testing method for detection, and the presence of low levels of pathogen especially at early stages of infection, threaten the effectiveness of these surveys. Therefore, new methods that increase detection efficiency and are applicable to field-based testing are urgently needed.

Changes to the leaf tissue at an anatomical level that correlate with infection by Las have been noted in citrus. These include middle lamella swelling/phloem necrosis, obstruction of sieve elements resulting from the deposition of callose and phloem protein 2, and disruption of chloroplast structure caused by starch accumulation. Although these changes appear to occur universally across the different citrus species upon infection and may act as a good indicator for presence of Las, visualization of these changes have been made by microscopy. Since microscopy is not a technique that is easily adaptable to rapid detection in the field, alternative approaches for identifying such changes may be more applicable to disease detection. For example, tissue elasticity has been shown to be a means by which cellular changes can be measured.

It has been observed that certain diseased plant tissues exhibit elasticities that differ from those of their healthy counterparts. For example, HLB-affected orange leaves appear stiffer than leaves from healthy trees. Currently, no HLB detection method has been developed that capitalizes on this observed mechanical difference. The method currently used, real-time polymerase chain reaction (RT-PCR) is expensive, requires experience to identify samples (based upon symptoms) that will not yield false-negative results due to inadequate amounts of bacterial DNA being present, relies upon destructive sampling methods, and is not commercially available in a format that can be used for detection in the field. Because the equipment needed to measure mechanical variances is greatly simplified compared to that required to perform PCR, detection devices based upon mechanical differences can be made compact and work with a battery as a power supply; allowing detection capabilities to be moved from a laboratory-based setting into a field setting where the diseased plants are located. Detector (sniffing) dogs are a more recent method for detection that can be used in the field. However, the sensitivity amongst dogs can differ significantly and dogs require rigorous training and a specialized handler; therefore, mass production is not readily achievable.

Thus, new methods for the rapid and sensitive screening of disease in plants are needed.

### SUMMARY OF THE DISCLOSURE

Provided herein is a soft leaf tissue elasticity tester, a device useful for obtaining measurements to quantify soft leaf tissue elasticity, to rapidly and sensitively screen plants for diseases by quantifying changes in tissue elasticity and/or stiffness resulting from a pathogen-induced diseased state within the plant. Also provided are methods for screening plants for diseases by quantifying changes in tissue elasticity and/or stiffness resulting from a pathogen-induced diseased state within the plant.

In an embodiment, the disclosure relates to a soft leaf tissue elasticity tester comprising a piezoelectric finger (PEF) and a sample pad, and optionally comprising a DC voltage and a voltage detector in communication with the PEF. In some embodiments of the disclosure, the PEF in the soft leaf tissue elasticity tester comprises a first piezoelectric layer having a first length and being configured for application of a voltage thereto; a second piezoelectric layer having a second length; an optional non-piezoelectric layer a proximal portion of which is located between the first piezoelectric layer and the second piezoelectric layer; and a probe having a first end opposite a second end, the first end coupled at a distal end of the first piezoelectric layer or the non-piezoelectric layer when present, and the second end having a contact area. In some embodiments of the disclosure, the non-piezoelectric layer is present.

In some embodiments of the disclosure, in the soft elasticity tester, the first piezoelectric layer, the second piezoelectric layer, and the non-piezoelectric layer when present, are coupled at a distal end from the probe. In some embodiments of the disclosure the probe of the soft leaf tissue elasticity tester has a width from about one hundred times smaller than the soft tissue sample thickness to as large as the sum of the thickness of the soft tissue sample and that of the sample pad. In some embodiments of the disclosure, the sample pad of the soft leaf tissue elasticity tester has a depth larger than at least about two times the width of the probe. In some embodiments of the disclosure, when the sample pad of the soft leaf tissue elasticity tester forms a contact with a soft leaf tissue, no detectable air pockets are formed between the sample pad and the soft leaf tissue. In some embodiments of the disclosure, the sample pad of the soft leaf tissue elasticity tester is a gel with an elastic modulus smaller than a calculated elastic modulus of a soft leaf tissue to be measured. In some embodiments of the disclosure, the sample pad of the soft leaf tissue elasticity tester has an elastic modulus from about 50 kPa to about 500 kPa. In some embodiments of the disclosure, the soft leaf tissue elasticity tester is one unit. In some embodiments of the disclosure, the one unit soft leaf tissue elasticity tester is automated.

In an embodiment, the disclosure relates to a method for screening a plant for disease by determining an elastic modulus of the soft leaf tissue from the plant, comparing the elastic modulus determined for the soft leaf tissue to a calculated elastic modulus cut-off, and determining that the plant is diseased if the determined elastic modulus is higher than the calculated clastic modulus cut-off. In some embodiments of the disclosure, the clastic modulus of the soft leaf tissue is determined from measurements obtained with a soft leaf tissue elasticity tester described herein. In some embodiments of the disclosure, the clastic modulus cut-off for a specific plant species is calculated from the determined elastic modulus for healthy and diseased leaf tissue from such a plant. In some embodiments of the disclosure, the clastic modulus cut-off is calculated to achieve at least about 80% sensitivity and at least about 80% specificity between healthy and diseased plant leaf tissue. In some embodiments of the disclosure, disease in the method for screening a plant for disease is a viral infection, a bacterial infection, or a fungal infection. In some embodiments of the disclosure the screened plant in the method for screening a plant for disease is a Rutaceae family member.

### BRIEF DESCRIPTION OF THE DRAWINGS

Advantages of the embodiments presented herewith will be apparent from the following detailed description of the exemplary embodiments. The following detailed description should be considered in conjunction with the accompanying Examples and figures in which:

FIG. 1 depicts a schematic diagram of an illustrative soft leaf tissue elasticity tester comprising a modified piezoelectric finger (PEF) and a sample pad holder described herein.

FIG. 2 shows a schematic diagram of illustrative one unit soft leaf tissue elasticity tester described herein.

FIG. 3 depicts a photograph of an exemplary soft leaf tissue elasticity tester. The PEF, sample pad (leaf holder), probe, and leaf samples are indicated.

FIG. 4A and FIG. 4B depict exemplary graphs used to calculate the clastic modulus. FIG. 4A shows a plot of the measured induced voltages with a sample (Vᵢₙ) and without a sample (V_{in,0}) vs the applied voltage (V). The Y Axis shows the induced voltage in millivolts (mV). The X axis shows the applied voltage (V). FIG. 4B shows the measured voltages arranged in a plot of (½)(π/A)^{1/2} (1−ν²) K(V_{in,0}−Vᵢₙ) versus Vᵢₙ. Where ν=0.5 is Poisson's ratio of the leaf sample, A is the contact area of the probe, and K=158 N/m is the effective spring constant of the PEF determined from a force versus displacement curve generated with weights and a Keyence laser displacement meter (Keyence) measurements. Filled squares (▪) indicate induced voltage without a sample (V_{in,0}), and filled circles (●) indicate induced voltage with a sample (Vᵢₙ).

FIG. 5A and FIG. 5B depict images of leaves and branches used with the soft leaf tissue elasticity tester described herein. FIG. 5A shows photographs of representative branches collected. FIG. 5B shows photographs of representative leaves, and of the rectangular leaf pieces excised from them to be tested with the soft leaf tissue elasticity tester described herein.

FIG. 6A to FIG. 6C depict graphs of the elastic modulus of grapefruit leaves at different stages of HLB infection, as determined from measurements obtained using the soft leaf tissue elasticity tester described herein. FIG. 6A shows the clastic modulus determined for grapefruit leaves at different stages of HLB infection. The Y axis shows the clastic modulus in kPa; the X axis shows the leaf types. Filled squares (▪) indicate data from healthy, Las⁻ (GFT-1G) leaves; horizontal arrows ( [symbol] ) indicate data from asymptomatic, Las⁺ (GFT-2G) leaves; upward facing triangles (▴) indicate data from Blotchy Mottle, Las⁺ (GFT-3G) leaves; downward facing triangles (▾) indicate data from Yellow, Las⁺ (GFT-4G) leaves. The dashed line shows the calculated grapefruit leaf elastic modulus cut-off. FIG. 6B shows the normal distribution of the clastic modulus values determined for Las (GFT-1G) and Las⁺ (GFT-2G, GFT-3G, and GFT-4G) grapefruit leaves. The Y axis shows the leaf count; the X axis shows the elastic modulus in KPa. Solid bars represent Las-leaves, striped bars represent Las⁺ leaves. The dashed line shows the calculated grapefruit leaf clastic modulus cut-off. FIG. 6C shows the receiver operating characteristic (ROC) curve for healthy vs. diseased grapefruit leaves.

FIG. 7A to 7C depict graphs of the elastic modulus of pummelo leaves at different stages of HLB infection, as determined from measurements obtained using the soft leaf tissue elasticity tester described herein. FIG. 7A shows the elastic modulus determined for pummelo leaves at different stages of HLB infection. The Y axis shows the clastic modulus in kPa; the X axis shows the leaf types. Filled squares (▪) indicate data from healthy, Las⁻ (PUM-1G) leaves; filled circles (●) indicate data from asymptomatic, Las⁺ (PUM-2G) leaves; upward facing triangles (▴) indicate data from symptomatic, Las⁺ (PUM-3G) leaves. The dashed line shows the calculated pumelo leaf clastic modulus cut-off. FIG. 7B shows the normal distribution of the clastic modulus values determined for Las⁻ (PUM-1G; solid bars) and Las⁺ (PUM-2G and PUM-3G; striped bars) pummelo leaves. The Y axis shows the pummelo leaf count; the X axis shows the clastic modulus in KPa. The dashed line shows the calculated pumelo leaf clastic modulus cut-off. FIG. 7C shows the receiver operating characteristic (ROC) curve for healthy vs. diseased pummelo leaves.

FIG. 8A to FIG. 8C depict graphs of the elastic modulus of lemon leaves at different stages of HLB infection, as determined from measurements obtained using the soft leaf tissue elasticity tester described herein. FIG. 8A shows the elastic modulus determined for lemon leaves at different stages of HLB infection. The Y axis shows the clastic modulus in kPa; the X axis shows the leaf types. Filled squares (▪) indicate data from healthy, Las⁻ (LEM-1G) leaves; filled circles (●) indicate data from asymptomatic, Las⁺ (LEM-2G) leaves; upward-facing triangles (▴) indicate data from symptomatic, Las⁺ (LEM-3G) leaves. The dashed line shows the calculated lemon leaf clastic modulus cut-off. FIG. 8B shows the normal distribution of the clastic modulus values determined for Las-(LEM-1G; solid bars) and Las⁺ (LEM-2G and LEM-3G; striped bars) lemon leaves. The Y axis shows the lemon leaf count; the X axis shows the clastic modulus in KPa. The dashed line shows the calculated lemon leaf elastic modulus cut-off. FIG. 8C shows the receiver operating characteristic (ROC) curve for healthy vs. diseased lemon leaves.

FIG. 9A to FIG. 9C depict graphs of the elastic modulus of Valencia sweet orange leaves at different stages of HLB infection, as determined from measurements using the soft leaf tissue elasticity tester described herein. FIG. 9A shows the clastic modulus determined for Valencia sweet orange leaves at different stages of HLB infection. The Y axis shows the elastic modulus in kPa; the X axis shows the leaf types. Filled squares (▪) indicate data from healthy, Las⁻ (VAL-1G) leaves; filled circles (●) indicate data from asymptomatic, Las⁺ (VAL-2G) leaves; upward-facing triangles (▴) indicate data from symptomatic, Las⁺ (VAL-3G) leaves; downward-facing triangles (▾) indicate data from Yellow, Las⁺ (VAL-4G) leaves. The dashed line shows the calculated Valencia sweet orange leaf elastic modulus cut-off. FIG. 9B shows the normal distribution of the elastic modulus values determined for Las-(VAL-1G; solid bars) and Las⁺ (VAL-2G and VAL-3G; striped bars) Valencia sweet orange leaves. The Y axis shows the Valencia sweet orange leaf count; the X axis shows the elastic modulus in KPa. The dashed line shows the calculated Valencia sweet orange leaf elastic modulus cut-off. FIG. 9C shows the receiver operating characteristic (ROC) curve for healthy vs. diseased Valencia sweet orange leaves.

### BRIEF DESCRIPTION OF THE SEQUENCE LISTING

The sequences disclosed in the specification and their sequence identifiers are listed in Table 1, below.

TABLE 1
SEQUENCES AND SEQUENCE IDENTIFIERS

| Identifier | Type | Name | Sequence |
|---|---|---|---|
| SEQ ID NO: 1 | DNA | Primer HLBasf | TCGAGCGCGTATGCGAATACG |
| SEQ ID NO: 2 | DNA | Primer HLBr | GCGTTATCCCGTAGAAAAAGGTAG |
| SEQ ID NO: 3 | DNA | Probe HLBp | AGACGGGTGAGTAACGCG |

### DETAILED DESCRIPTION

The present disclosure relates to a soft leaf tissue elasticity tester comprising a piezoelectric finger (PEF) sensor and a sample pad, useful for rapidly and sensitively screening plants for disease by quantifying changes in soft leaf tissue elasticity or stiffness. The disclosure also relates to the use of the soft leaf tissue elasticity tester described herein for the rapid and sensitive screening of plants for diseases by quantifying changes in soft leaf tissue elasticity or stiffness.

Prior to the instant application, elasticity of plant leaves has been used to measure tolerance to abiotic stresses such as drought, by comparing data from leaf tissues across entirely different species. However, elasticity of soft leaf tissue has not previously been used for screening diseased plant leaves.

Described herein is a soft leaf tissue elasticity tester created by modifying an existing piezoelectric finger (PEF) sensor and using a sample holder. This soft leaf tissue elasticity tester was used to determine the clastic modulus of different types of HLB infected and non-infected citrus leaves. In the instant disclosure, results from four different types of citrus demonstrated a direct correlation between the determined clastic modulus and the presence of disease. In the instant disclosure, measurements obtained with the soft leaf tissue elasticity tester were used to determine the elastic modulus of a tissue sample and ascertain the presence of huanglongbing (HLB). Using the soft leaf tissue elasticity tester described herein, differences were observable between the elastic modulus determined for low-titer, asymptomatic leaves and those determined for healthy (non-infected) leaves. This is critical for early-stage detection, and demonstrates the utility of the use of mechanical measurements as an early screening method for HLB.

The measurements obtained were also quantitative, thus, data obtained with the soft leaf tissue elasticity tester described herein may be assessed and tracked using predictive mathematical modeling.

Use of the soft leaf tissue elasticity tester described herein as a screening platform for HLB has many advantages over the current industry standard of RT-PCR-based methods. For example, the soft leaf tissue elasticity tester does not require expensive equipment to operate, nor are consumables required for testing. In addition, the simplicity of the soft leaf tissue elasticity tester described herein eliminates the need for highly-trained personnel to perform the testing, expanding testing capabilities to include citrus producers and/or field workers, which will ultimately reduce testing costs on a per sample basis. Because this technology also allows testing to be performed in the field and yields results almost instantaneously, it decreases the time-to-results, which is one of the most important factors for preventing HLB spread. Testing plants in the field may be done by cutting leaf sections and performing the measurements at a desk, or by placing the soft leaf tissue elasticity tester on a flat surface. It is also possible for one or two people to hold the leaf tissue elasticity tester. The modified PEF may be held one hand, and the sample pad or sample holder containing the sample pad on another hand. The two hands may belong to one single individual, or there may be two different individuals holding the different parts of the soft leaf tissue elasticity tester. Furthermore, the soft leaf tissue elasticity tester may form one unit comprising a modified PEF held together with a sample pad or sample pad holder containing a sample pad.

The soft leaf tissue elasticity tester described herein may be used to ascertain the presence or absence of disease in plants. Even if used only as an HLB screening device, the soft leaf tissue elasticity tester described herein will help save resources by eliminating the large number of HLB-negative samples currently being tested by RT-PCR.

Because the soft leaf tissue elasticity tester described herein may utilize a non-destructive sampling method, i.e., only one branch or leaf is taken from plants in the field, or small plants are tested in the greenhouse. Thus, the leaf tissue elasticity tester described will also be useful for identifying new cultivars that are either resistant to HLB or show tolerance to HLB. Plants in the greenhouse are small and have fewer leaves, so testing leaves still on the plant will allow testing small plants multiple times within a short window of time. These cultivars are a critical component to reviving the citrus industry in areas where HLB has become endemic such as the Southeastern portion of the United States. Additionally, the soft leaf tissue elasticity tester described herein will allow new therapeutics to be evaluated at much earlier stages than present detection methods permit, resulting in both time and cost-savings.

As seen in the Examples below, the determined elastic modulus values and the calculated elastic modulus cut-off differed depending upon the type of citrus tested. These differences can be accounted for when assessing leaf stiffness via the soft leaf tissue elasticity tester described herein. The calculated leaf elastic modulus cut-off for each leaf type was determined as the value at which the positive and negative fitted Gaussian distribution curves overlapped. It may be possible to incorporate a pull-down menu for each plant type into an automated testing software. This pull-down menu would include the calculated elastic modulus cut-off for the plant being tested. The elastic modulus cut-off may be calculated to achieve a desired sensitivity and specificity between healthy and diseased soft leaf tissues. For example, it may be desirable that the specificity of the calculated elastic modulus cut-off be from about 80% to about 99%, or any number in between. Similarly, it is desirable that the sensitivity of the calculated elastic modulus cut-off be from about 80% to about 99%, or any number in between. As calculated herein, the elastic modulus cut-off for grapefruit leaves achieved a 93% sensitivity and an 89% specificity, for pumelo leaves achieved a 96% sensitivity and a 95% specificity, for lemon leaves achieved an 89% sensitivity and an 84% specificity of, and for Valencia oranges achieved a 95% sensitivity and an 89% specificity. The calculated leaf elastic modulus cut-off for each soft leaf tissue type may be determined as the value at which the positive and negative fitted Gaussian distribution curves overlap.

In an embodiment of the invention, a hand-held device may be used for determining differences in plant tissue elasticity in combination with the methodology taught herein to determine the elastic modulus of a plant. The elastic modulus determined may be compared to a calculated clastic modulus cut-off specific for each type of plant to ascertain the presence or absence of disease in the plant. The hand-held device may be a soft leaf tissue elasticity tester comprising a modified piezoelectric finger (PEF) sensor and a sample pad as taught herein. A PEF is a cantilever structure with an apparatus on the tip to control the contact area and two or more layers of piezoelectric materials. When a DC voltage is applied to one piezoelectric layer, it induces a deflection in the cantilever structure. The deflection of the cantilever imparts a force upon the surface in which a probe makes contact. The deflection of the cantilever structure induces a voltage in a second, electronically separated piezoelectric transducer within the cantilever, which can be used to determine the displacement. The orientation of the applied force relative to a substrate can be varied so that the applied force can be normal or parallel to the surface of the material to be assessed. The contact area of the device can be controlled so that the applied stress can be calculated, and the displacement can be used to quantify the induced strain. Using the applied stress and measured strain, the Young's or shear modulus of the material can be determined.

The clastic modulus may be determined by placing the PEF probe on top of the object to be measured (the sample holder or a tissue section located on top of the sample holder) and applying a voltage to the driving PZT layer using a function generator. This voltage will exert a force on the object which in turn induces a piezoelectric voltage across the sensing PZT layer. Outputs may be recorded using an oscilloscope, and the elastic modulus determined using the following mathematical equation:

$$E = ( 1 / 2 ) {( π / 2 )}^{1 / 2} ( 1 - {v}^{2} ) K ( Vin 0 - Vin ) / Vin ) \qquad Equation 1$$

where V_{in,0} and Vᵢₙ are the induced voltages without and with the tissue, respectively, ν is Poisson's ratio of the tissue (which in this case is equal to 0.5 due to the incompressibility of water contained in the tissue), A is the contact area of the probe, and K is the effective spring constant of the PEF 19.

Through control of the contact area, the stiffness of the sample pad, the duration of the applied force, and the length of the cantilever, the output of the amount of applied stress and the sensitivity measured due to the induced strain can be well controlled. This ultimately yields a device capable of performing measurements sensitive enough to allow the calculation of the elastic modulus of a soft leaf tissue for the implementation of the process taught herein. In addition, the modified PEF in the soft leaf elasticity tester described herein can be fabricated with an array of different cantilevers and used to scan a defined area producing a 2D stiffness map of the entire sample. The use of the modified PEF in the soft leaf elasticity tester described herein in combination with the methods described herein may be ideal since the soft leaf elasticity tester described herein can be easily integrated into a hand-held device. One such example of a handheld PEF is the iBreastExam® (registered to UE Lifesciences, Inc.; Philadelphia, Pennsylvania, USA); a 16-cantilever array, hand-held device that has been FDA approved for assessing the stiffness of breast tissue for breast cancer screening (V. L. Mango, et al., 2022, “The iBreastExam versus clinical breast examination for breast evaluation in high risk symptomatic Nigerian women: a prospective study,” Lancet Glob. Health 2022; 10: e555-63).

In an embodiment, the disclosure relates to a soft leaf elasticity tester comprising a modified piezoelectric finger (PEF) sensor and a sample pad. The soft leaf tissue elasticity tester described herein is useful for determining the elastic modulus of soft leaf tissue and detecting disease in plants. Prior to the present disclosure, a PEF had not been used with a sample pad to detect disease on plant leaves. Prior use of a PEF was constrained to identifying abnormal cells (i.e.: tumors) within human tissues. Due to the thin and delicate nature of most soft leaf tissue, the soft leaf tissue elasticity tester comprises a PEF and a sample stage/sample pad onto which the soft leaf tissue to be measured is placed for interrogation by the PEF. In addition, the PEF for tumor detection has a probe that is 7 mm or wider, to ensure that it can sample breast tissues deep enough underneath the surface to catch small tumors deep under the surface. The modified PEF in the soft leaf tissue elasticity tester described herein has a very thin probe to contact the leaf sample, that is about half the size of the thickness of the soft leaf tissue to be tested.

The depth sensitivity of the PEF elasticity sensor is known to be twice the diameter of the probe contacting the material being tested. While a very thin probe is more likely to produce an elastic modulus close to that of the leaf, it is also more likely to poke into the leaf and produce erroneous elastic modulus measurements. To avoid such pitfalls, probes with a diameter larger than the leaf thickness were used where a PEF measurement assesses not only the elasticity of the leaf but also part of the leaf sample holder under the leaf. To prevent the measured elastic moduli from being overwhelmed by a hard sample holder, a gelatinous leaf sample holder that was softer than the leaf samples was utilized. The sample holder consisted of a gelatin gel stored in a petri dish sealed with paraffin. When in use, the gelatin gel was flipped over so that the bottom side made contact with the leaf samples. In the Examples described herein, the soft leaf tissue elasticity tester used to determine the elastic modulus of leaves had a probe of a width of about 0.2 mm or less. This is because the depth of tissues that will contribute to a PEF clastic modulus determination is roughly twice the width of the probe.

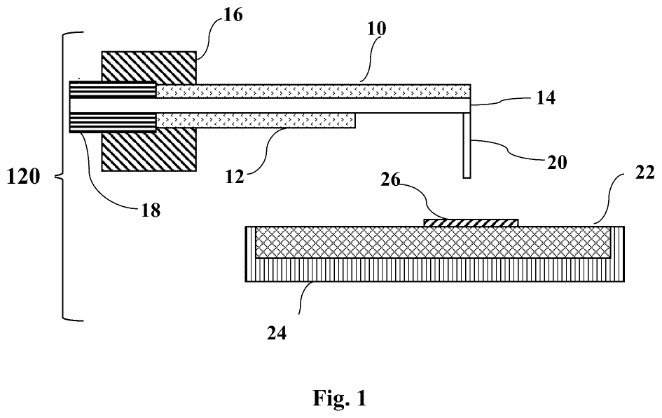

As shown in FIG. 1, the soft leaf tissue elasticity tester 120 comprises a modified piezoelectric finger (PEF) with two piezoelectric layers, a top sensing piezoelectric layer 10 and a bottom driving piezoelectric layer 12, and a sample pad 22. The top piezoelectric layer 10 and the bottom piezoelectric layer 12 may be made of lead zirconate titanate, barium titanate, gallium nitride, lead magnesium niobate, lead titanate solid solution, zinc oxide, or any suitable piezoelectric material. A non-piezoelectric layer 14 may be located between the top sensing piezoelectric layer 10 and the bottom driving piezoelectric layer 12. This non-piezoelectric layer 14 may be made of stainless steel, or any other suitable non-piezoelectric material. The modified PEF further comprises a probe 20, and optionally may comprise a clamp 16 and a spacer 18. The clamp may be located at one end of the modified PEF, and the probe may be at a distal end from the clamp. The system 120 further may comprise an optional sample pad holder 24 holding the sample pad 22. The schematic shows a sample 26 on the sample pad 22. The modified PEF is in electronical communication with a means for applying an electrical current, a computer processor and associated display through connectors. Any means of electronic communication known in the art may be used for the connectors, such as wires, or wireless communication.

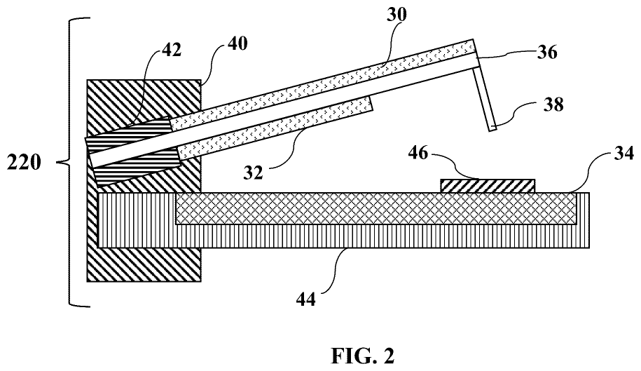

As shown in FIG. 2 the soft leaf tissue elasticity tester 220 comprising a modified piezoelectric finger (PEF) with two piezoelectric layers, a top sensing piezoelectric layer 30 and a bottom driving piezoelectric layer 32, and a sample pad 34 may be forming a single unit held by a clamp 40. The top piezoelectric layer 30 and the bottom piezoelectric layer 32 may be made of lead zirconate titanate, barium titanate, gallium nitride, lead magnesium niobate, lead titanate solid solution, zinc oxide, or any suitable piezoelectric material. A non-piezoelectric layer 36 may be optionally located between the top sensing piezoelectric layer 30 and the bottom driving piezoelectric layer 32. This non-piezoelectric layer 36 may be made of stainless steel, or any other suitable non-piezoelectric material. The modified PEF further comprises a probe 38, and optionally a spacer 42. The clamp is located at one end of the modified PEF, and the probe is at a distal end. The system 220 further may comprise an optional sample pad holder 44 holding the sample pad 34. The schematic shows a sample 46 on the sample pad 34. The modified PEF is in electronical communication with a means for applying an electrical current, a computer processor and associated display through connectors. Any means of electronic communication known in the art may be used for the connectors, such as wires, or wireless communication.

As used herein, “top piezoelectric layer” and “first piezoelectric layer” are used interchangeably. Similarly, “bottom piezoelectric layer” and “second piezoelectric layer” are used interchangeably. The first piezoelectric layer has a first length, and the second piezoelectric layer has a second length that may be shorter than, equal to, or longer than the first length. In some embodiments of the disclosure the first piezoelectric layer, the second piezoelectric layer, and the non-piezoelectric layer when present are joined with a clamp at one end.

As used herein, the terms “sample pad”, “stage”, and “leaf holder” are used interchangeably, and refer to a surface upon which a leaf is placed to perform measurements to determine its elastic modulus.

As used herein, the terms “coupled” and “joined” are used interchangeably, and refer to parts of the PEF held in place by any way known in the art. For example, the parts of the PEF may be held in place by an adhesive or a clamp.

Due to the delicate nature of the soft leaf tissue, the sample must be placed upon a sample pad/stage to obtain measurements. The sample pad may be composed of any soft gel with an elastic modulus less than that of the elastic modulus determined for the soft leaf tissue to be measured. To assist the user, a tester spec sheet may be provided with the soft leaf tissue elasticity tester listing the soft leaf tissue elastic modulus cut-off for different plant types. This is to ensure the elasticity measurement is mainly due only to the soft leaf tissue. The soft sample pad may be made of any material that can form a gel such as polydimethylsiloxane (PDMS), silicone, or gelatin. The thickness of the sample pad (d) must be such that it is more than about two times the size of the probe (w). The probe should be less than about 4 times the thickness of the soft leaf tissue (t). In some embodiments, the probe is about 2 mm. The sample pad material should not shrink. It is important for the sample pad to be dimensionally stable.

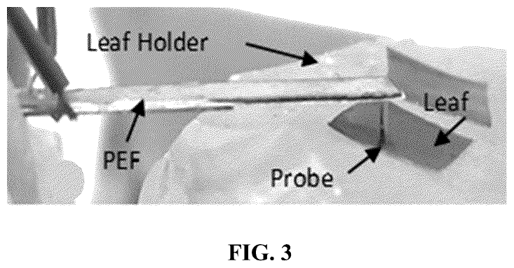

A photograph of a possible soft leaf tissue elasticity tester is shown in FIG. 3. An electronic current applied across the driving piezoelectric layer of the modified PEF will generate an axial displacement resulting in an axial force at the tip of the probe allowing the probe to contact a soft leaf tissue placed on the sample pad/stage. This resulting electronic signal is transmitted to a computer processor and associated display.

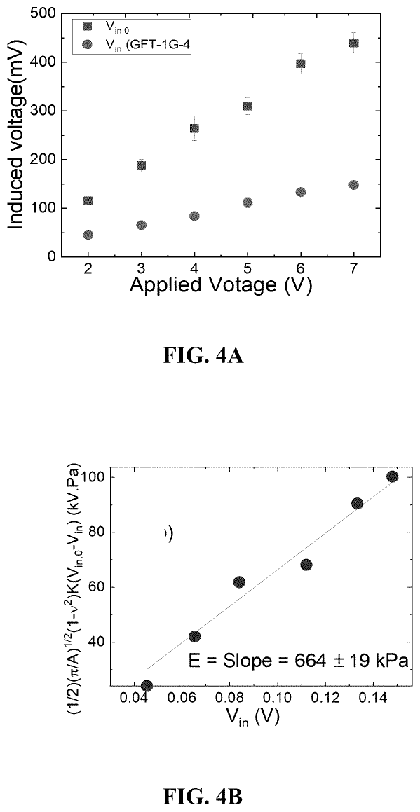

Graphs used to calculate the clastic modulus are shown in FIG. 4A and FIG. 4B. FIG. 4A shows a plot of the measured induced voltages with a sample (Vᵢₙ) and without a sample (V_{in,0}) vs the applied voltage (V). FIG. 4B shows the measured voltages arranged in a plot of (½)(π/A)^{1/2}(1−ν²)K(V_{in,0}−Vᵢₙ) versus Vᵢₙ. Where ν=0.5 is Poisson's ratio of the leaf sample, A is the contact area of the probe, and K=158 N/m is the effective spring constant of the PEF determined from a force versus displacement curve generated with weights and a Keyence laser displacement meter (Keyence) measurements.

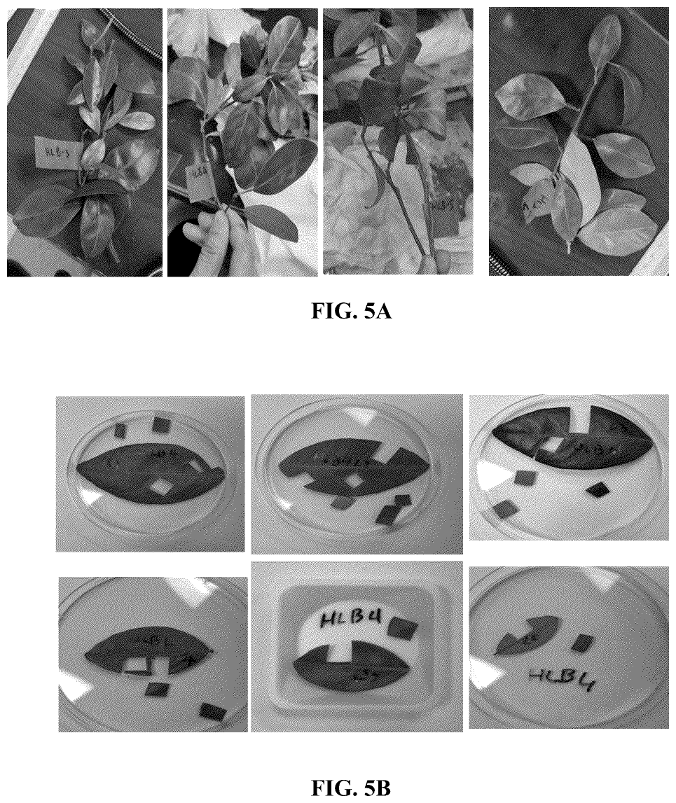

Photographs of representative branches and leaves collected to be tested using the soft leaf tissue elasticity tester described herein are shown in FIG. 5A. Photographs of representative leaves and representative rectangular leaf pieces excised to be tested with the soft leaf tissue elasticity tester described herein are shown in FIG. 5B.

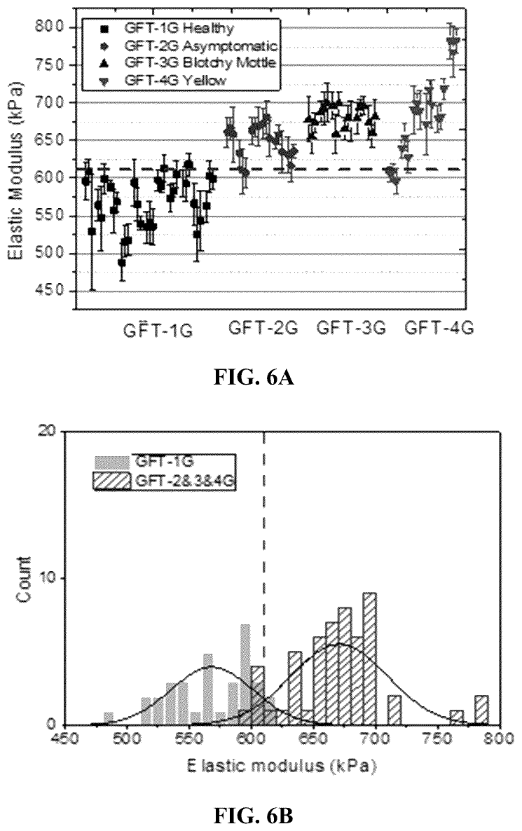

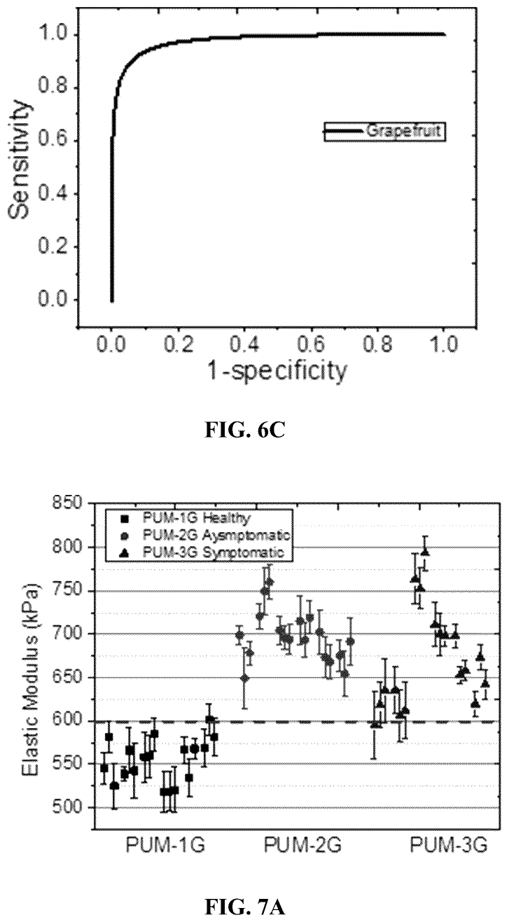

The inventors obtained grapefruit and Valencia orange leaves that appeared healthy, asymptomatic, blotchy mottle, or yellow; and pummelo and lemon leaves that appeared healthy, asymptomatic, or symptomatic, and determined their clastic modulus using the soft leaf tissue elasticity tester described herein. As seen in FIG. 6A and FIG. 6B, the clastic modulus determined for grapefruit leaves at different stages of HLB infection segregate according to the HLB infection stage. In FIG. 6A the Y axis shows the mean and standard deviation of the clastic modulus determined in kPa, and the X axis shows the leaf types: filled squares (▪) indicate data from healthy, Las⁻ (GFT-1G) leaves; horizontal arrows ( [symbol] ) indicate data asymptomatic, Las⁺ (GFT-2G) leaves; upward facing triangles (▴) indicate data from Blotchy Mottle, Las⁺ (GFT-3G) leaves; and downward facing triangles (7) indicate data from Yellow, Las⁺ (GFT-4G) leaves. The dashed line shows the calculated grapefruit leaf clastic modulus cut-off. FIG. 6B shows the normal distribution of the elastic modulus values determined for Las-(GFT-1G) and Las⁺ (GFT-2G, GFT-3G, and GFT-4G) grapefruit leaves. The Y axis shows the leaf count; the X axis shows the clastic modulus in KPa. Solid bars represent Las-leaves, striped bars represent Las⁺ leaves. Using the values in these two graphs, a grapefruit-specific clastic modulus cutoff of 611 kPa was deduced to achieve a 93% sensitivity and a 89% specificity between healthy and diseased grapefruit leaves. This calculated grapefruit-specific elastic modulus cut-off is indicated by dashed bars in FIG. 6A and FIG. 6B. As can be seen in these two figures, the calculated grapefruit-specific clastic modulus cut-off separated the majority of the healthy leaves from the infected leaves. FIG. 6C shows the receiver operating characteristic (ROC) curve for healthy vs. diseased grapefruit leaves. For GFT-1G, eleven different samples were tested in triplicates whereas for GFT-2G, GFT-3G and GFT-4G, six different samples were tested in triplicate with error bars representing the standard deviation of the measurement.

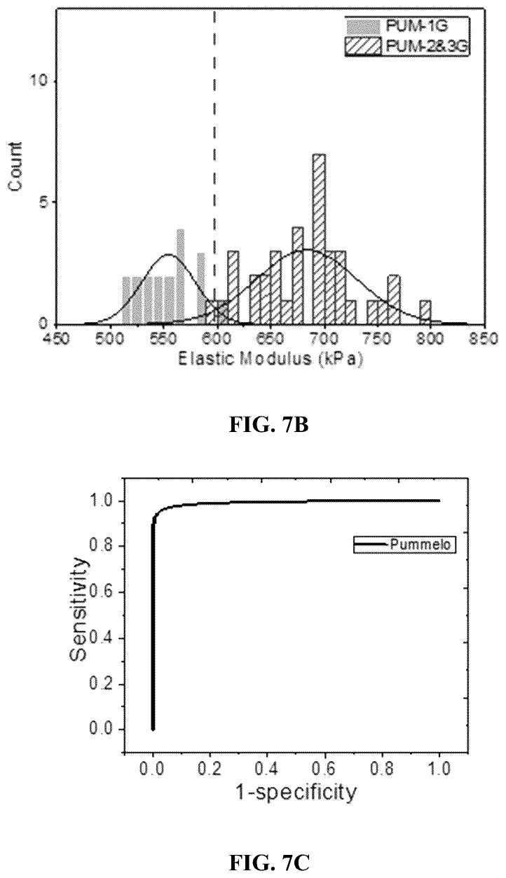

As seen in FIG. 7A and FIG. 7B, the clastic modulus determined for pumelo leaves at different stages of HLB infection, as measured using the soft leaf tissue elasticity tester described herein, segregate according to the HLB infection stage. In FIG. 7A the Y axis shows the mean and standard deviation of the determined clastic modulus in kPa, and the X axis shows the types of leaves: filled squares (▪) indicate data from healthy, Las⁻ (PUM-1G) leaves; filled circles (●) indicate data from asymptomatic, Las⁺ (PUM-2G) leaves; upward facing triangles indicate data from symptomatic, Las⁺ (PUM-3G) leaves. FIG. 7B shows the normal distribution of the clastic modulus values determined for Las-(PUM-1G) and Las⁺ (PUM-2G and PUM-3G) pummelo leaves. The Y axis shows the pummelo leaf count; the X axis shows the deduced clastic modulus in KPa. Solid bars represent Las⁻ pummelo leaves, striped bars represent Las⁺ pummelo leaves. Using the values in these two graphs, an elastic modulus cutoff of 598 kPa was deduced to achieve a 96% sensitivity and a 95% specificity between healthy and diseased grapefruit leaves. This calculated pumelo-specific clastic modulus cut-off is indicated by dashed bars in FIG. 7A and FIG. 7B. As can be seen in these two figures, the calculated pumelo-specific elastic modulus cut-off separated the majority of the healthy leaves from the infected leaves. FIG. 7C shows the receiver operating characteristic (ROC) curve for healthy vs. diseased pummelo leaves. For PUM-1G, PUM-2G and PUM-3G six different samples were tested in triplicate with the error bars representing the standard deviation of the measurements.

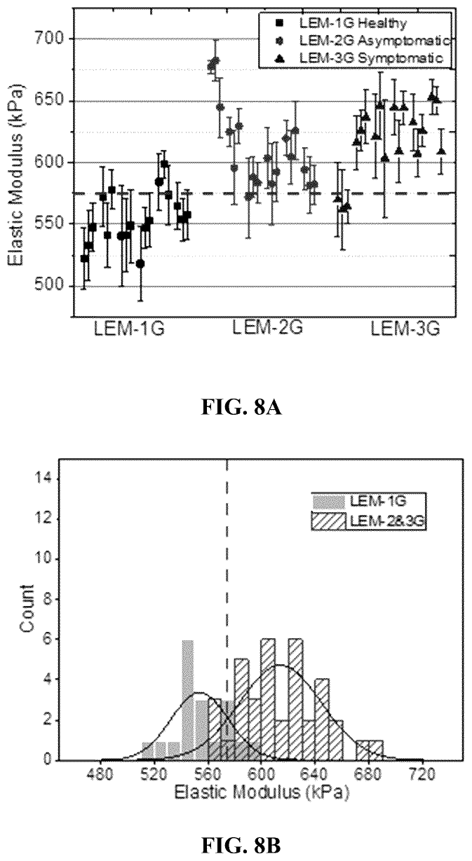

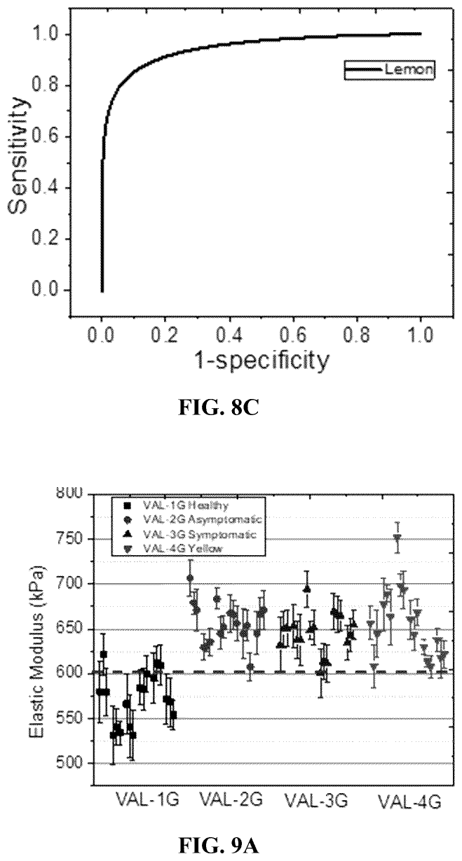

As seen in FIG. 8A and FIG. 8B, the calculated elastic modulus of lemon leaves at different stages of HLB infection, as measured using the soft leaf tissue elasticity tester described herein, segregate according to the HLB infection stage. In FIG. 8A, the Y axis shows the mean and standard deviation of the determined clastic modulus in kPa; the X axis shows the types of leaves: filled squares (▪) indicate data from healthy, Las⁻ (LEM-1G) leaves; filled circles (●) indicate data from asymptomatic Las⁺ (LEM-2G) leaves; upward-facing triangles (▴) indicate data from symptomatic, Las⁺ (LEM-3G) leaves. FIG. 8B shows the normal distribution of the elastic modulus values determined for Las-(LEM-1G) and Las⁺ (LEM-2G and LEM-3G) lemon leaves. The Y axis shows the lemon leaf count; the X axis shows the elastic modulus in KPa. Solid bars represent Las-lemon leaves, striped bars represent Las⁺ lemon leaves. Using the values in these two graphs, an elastic modulus cutoff of 575 kPa was deduced to achieve a 89% sensitivity and a 84% specificity between healthy and diseased lemon leaves. This calculated lemon-specific elastic modulus cut-off is indicated by dashed bars in FIG. 8A and FIG. 8B. As can be seen in these two figures, the calculated lemon-specific elastic modulus cut-off separated the majority of the healthy leaves from the infected leaves. FIG. 8C shows the receiver operating characteristic (ROC) curve for healthy vs. diseased lemon leaves. For LEM-1G, LEM-2G and LEM-3G six different samples were tested in triplicate with error bars representing the standard deviation of the measurement.

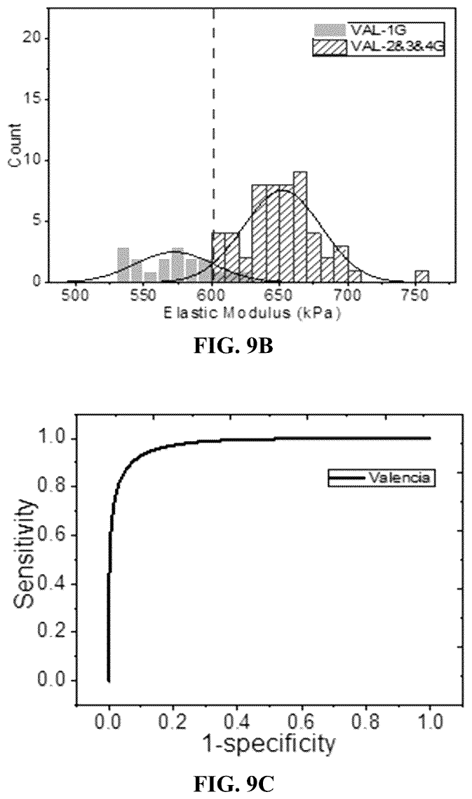

As seen in FIG. 9A and FIG. 9B, the elastic modulus of Valencia sweet orange leaves at different stages of HLB infection, as measured using the soft leaf tissue elasticity tester described herein, segregate according to the HLB infection stage. In FIG. 9A the Y axis shows the mean and standard deviation of the determined elastic modulus in kPa; the X axis shows the types of leaves: filled squares (▪) indicate data from healthy, Las⁻ (VAL-1G), leaves; filled circles (●) indicate data from asymptomatic, Las⁺ (VAL-2G), leaves; upward-facing triangles (▴) indicate data from symptomatic, Las⁺ (VAL-3G),) leaves; downward-facing triangles (▾) indicate data from Yellow, Las⁺ (VAL-4G) leaves. FIG. 9B shows the normal distribution of the clastic modulus values determined for Las-(VAL-1G) and Las⁺ (VAL-2G and VAL-3G) Valencia sweet orange leaves. The Y axis shows the Valencia sweet orange leaf count; the X axis shows the elastic modulus in KPa. Solid bars represent Las-sweet orange leaves, striped bars represent Las⁺ sweet orange leaves. Using the values in these two graphs, an elastic modulus cutoff of 602 kPa was deduced to achieve a 95% sensitivity and a 84% specificity between healthy and diseased Valencia sweet orange leaves. This calculated Valencia sweet orange-specific clastic modulus cut-off is indicated by dashed bars in FIG. 9A and FIG. 9B. As can be seen in these two figures, the calculated Valencia sweet orange-specific elastic modulus cut-off separated the majority of the healthy leaves from the infected leaves. FIG. 9C shows the receiver operating characteristic (ROC) curve for healthy vs. diseased Valencia sweet orange leaves. For VAL-1G, VAL-2G, VAL-3G and VAL-4G six different samples were tested in triplicate with error bars representing the standard deviation of the measurement.

Because of this ability to identify HLB-affected leaves using elastic modulus determined from measurements obtained using the soft leaf tissue elasticity tester described herein prior to the onset of symptoms, the soft leaf tissue elasticity tester described herein, comprising a modified PEF and a sample pad, may be used as an early screening method for HLB. Additionally, measurements of elasticity or stiffness may also be applied for the evaluation of resistant/tolerant citrus germplasm in the greenhouse regardless of Candidatus Liberibacter asiaticus (Las) bacterial titers; ultimately decreasing the time/space needed for cultivar screening and evaluation in the fields. It is also important to note that because leaves from HLB-affected plants have a measurable increase in leaf stiffness compared to healthy counterparts, detection of HLB can be differentiated from other abiotic stressors such as drought, which results in a loss of turgor pressure that leads to a decrease in leaf stiffness.

Citrus greening, or HLB, affects all citrus varieties and some ornamental plants, including boxwood and orange jasmine. Plants currently known at risk of HLB infection include Chinese box-orange, curry leaf, finger-lime, grapefruit, key lime, kumquat, lemon, lime, limeberry, mandarin orange, mock orange, orange, orange jasmine, pomelo, sour orange, sweet orange, tangerine, and trifoliate orange.

As used herein, the term “about” is defined as plus or minus ten percent of a recited value. For example, about 1.0 g means 0.9 g to 1.1 g.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

Thus, the following are described at least in part:

A soft leaf tissue elasticity tester comprising a piezoelectric finger (PEF) and a sample pad, and optionally comprising a DC voltage and a voltage detector in communication with the PEF.

The above soft leaf tissue elasticity tester, wherein the PEF comprises a first piezoelectric layer having a first length and being configured for application of a voltage thereto; a second piezoelectric layer having a second length; an optional non-piezoelectric layer, a proximal portion of which is located between the first piezoelectric layer and the second piezoelectric layer; and a probe having a first end opposite a second end, the first end coupled at a distal end of the first piezoelectric layer or the non-piezoelectric layer when present, and the second end having a contact area.

The above soft leaf tissue elasticity tester, wherein the first piezoelectric layer, the second piezoelectric layer, and the non-piezoelectric layer when present, are coupled at a distal end from the probe.

The above soft leaf tissue elasticity tester, wherein the non-piezoelectric layer is present.

The above soft leaf tissue elasticity tester, wherein the first piezoelectric layer, the second piezoelectric layer, and the non-piezoelectric layer are coupled at a distal end from the probe.

The above soft leaf tissue elasticity tester, wherein the probe width is from about one hundred times smaller than the soft leaf tissue sample thickness to as large as the sum of the thickness of the soft tissue sample and that of the sample pad.

The above soft leaf tissue elasticity tester, wherein the sample pad has a depth larger than at least about two times the width of the probe.

The above soft leaf tissue elasticity tester, wherein the sample pad is a gel with an elastic modulus smaller than a calculated elastic modulus of a soft leaf tissue to be measured.

The above soft leaf tissue elasticity tester, wherein a proximal portion of the sample pad is coupled to a proximal portion of the PEF.

The above soft leaf tissue elasticity tester, wherein a proximal portion of the first piezoelectric layer, the second piezoelectric layer, and the non-piezoelectric layer when present are coupled to a proximal portion of the sample pad.

The above soft leaf tissue elasticity tester, wherein the non-piezoelectric layer is present; and a proximal portion of the first piezoelectric layer, the second piezoelectric layer, and the non-piezoelectric layer are coupled to a proximal portion of the sample pad.

Any one of the above-described soft leaf tissue elasticity testers, wherein the soft leaf tissue elasticity tester is automated.

A method for screening a plant for disease, the method comprising: determining an elastic modulus of a soft leaf tissue from the plant; comparing the elastic modulus determined for the soft leaf tissue from the plant to a calculated elastic modulus cut-off; and concluding that the plant is diseased if the elastic modulus determined for the soft leaf tissue from the plant is larger than the calculated elastic modulus cut-off.

The above method for screening a plant for disease, wherein the elastic modulus cutoff is calculated to achieve at least about 80% sensitivity and at least about 80% specificity between healthy and diseased plant leaf tissue.

The above method for screening a plant for disease, wherein the elastic modulus is determined using measurements obtained with the soft leaf tissue elasticity tester comprising a PEF and a sample pad, and optionally comprising a DC voltage and a voltage detector in communication with the PEF.

The above method for screening a plant for disease, wherein the elastic modulus is determined using measurements obtained with the soft leaf tissue elasticity tester comprising a PEF and a sample pad, and optionally comprising a DC voltage and a voltage detector in communication with the PEF; wherein a proximal portion of the sample pad is coupled to a proximal portion of the PEF.

The above method for screening a plant for disease, wherein the elastic modulus is determined using measurements obtained with a soft leaf tissue elasticity tester comprising a PEF and a sample pad; and optionally comprising a DC voltage and a voltage detector in communication with the PEF; wherein the PEF comprises: a first piezoelectric layer having a first length and being configured for application of a voltage thereto; a second piezoelectric layer having a second length; an optional non-piezoelectric layer, a proximal portion of which is located between the first piezoelectric layer and the second piezoelectric layer; and a probe having a first end opposite a second end, the first end coupled at a distal end of the first piezoelectric layer or the non-piezoelectric layer when present, and the second end having a contact area.

The above method for screening a plant for disease, wherein the disease is a viral infection, a bacterial infection, or a fungal infection.

The above method for screening a plant for disease, wherein the disease is a bacterial infection, and the bacterial infection is Huanglongbing (HLB).

The above method for screening a plant for disease, wherein the screened plant is a member of the Rutaceae family.

The above soft leaf tissue elasticity tester, wherein the PEF consists of a driving piezoelectric layer and a sensing piezoelectric layer sandwiching a stainless-steel layer in a cantilever structure with a thin rod-shaped metal probe having a first end opposite a second end, the first end coupled at a distal end of the sensing piezoelectric layer for contacting the sample.

### EXAMPLES

Having now generally described this invention, the same will be better understood by reference to certain specific examples, which are included herein only to further illustrate the invention and are not intended to limit the scope of the invention as defined by the claims.

### Example 1

### Materials and Methods

The materials and methods used in the instant invention are described in this example.

Citrus leaf samples Citrus trees were grown within a greenhouse located at the US Horticultural Research Laboratory in Fort Pierce, Florida, USA. In an effort to preserve the integrity of leaves and keep leaves from unintentionally drying out, branches containing multiple leaves were removed from trees and stored at 4° C. until testing. FIG. 5A depicts photographs of stored branches. For testing, leaves were first separated from the branches and small (0.5 mm×0.5 mm) square segments were cut from the leaves, with their physical dimensions measured by calipers (FISHERBRAND TRACEABLE; Fisher Scientific Company LLC, and Control 3, LLC registered trademarks). As seen in FIG. 5B, these segments were removed from the edges and corners of the respective leaves. This area of the leaf is relatively flat compared to the leaf mid-vein. For symptomatic leaves, segments were taken from areas of the leaf that looked visibly diseased (yellowed or mottled).

Leaf samples were subsequently placed face down on with the adaxial side of the leaf against the gel sample pad/stage to minimize potential air pockets between the leaf and sample pad since air pockets can erroneously reduce the value of the measured elasticity modulus and cause a false-negative prediction. The gel sample pad had an elastic modulus smaller than that of a leaf such that the measured clastic modulus was mainly due to the leaf tissues.

Samples were obtained from four of the major citrus species/hybrids: 1) Citrus x paradisi noted as Grapefruit (GFT), 2) Citrus x sinensis noted as Valencia (VAL), 3) Citrus x limon noted as Lemon (LEM), and 4) Citrus maxima noted as Pummelo (PUM). Grapefruit samples were divided into four subgroups: healthy (GFT-1G), asymptomatic (GFT-2G), blotchy mottle (GFT-3G), and yellow (GFT-4G). Valencia samples were divided into four subgroups: healthy (VAL-1G), asymptomatic (VAL-2G), symptomatic (VAL-3G), and yellow (VAL-4G). Lemon samples were divided into three subgroups: healthy (LEM-1G), asymptomatic (LEM-2G), and symptomatic (LEM-3G). Pummelo samples were divided into three subgroups: healthy (PUM-1G), asymptomatic (PUM-2G), and symptomatic (PUM-3G). Six leaves ranging from young (small) to adult (long) were tested for each group, for each leaf the length and width was measured using a ruler and a picture taken using an iPhone 12 (iPhone is an Apple Inc. registered trademark) handheld mobile digital electronic device for sending and receiving digital data.

Fabrication of the modified piezoelectric finger (PEF) The PEF utilized was a 22 mm long and 4 mm wide cantilever consisting of a top piezoelectric layer for driving, a middle layer for mechanical support, and a bottom piezoelectric layer for sensing as shown in FIG. 1. Both the driving and sensing piezoelectric layers were composed of 127-μm thick lead zirconate titanate (PZT) (5H, T105-H4NO-2929; Piezo.com; Woburn, Massachusetts, USA) with the driving PZT being 22 mm long and 4 mm wide while the sensing PZT was 12 mm and 4 mm wide. The middle support layer was composed of 50 μm thick stainless-steel (Alfa Aesar; Haverhill, Massachusetts, USA). For leaf testing, a 4 mm long brass rod (Albion Alloys; Bournemouth, Bournemouth, United Kingdom) with a 0.4 mm diameter was glued to the stainless-steel side (underside) of the free end of the cantilever using a nonconductive epoxy to serve as the “probe” of the PEF. A photograph of the soft leaf elasticity tester used is shown in FIG. 3. Wires were soldered to the PEF, and it was subsequently clamped into an acrylic clamp (McMaster-Carr; Elmhurst, Illinois, USA) to fulfill the cantilever geometry with a defined length of 22 mm. A direct current (DC) voltage was applied using a DC power supply. The entire assembly was then affixed on a XYZ positioner (OptoSigma) using a C-clamp (Pony Jorgensen; Saddle Brook, New Jersey, USA).

Elastic modulus measurements using the PEF Prior to determining the elastic modulus of the citrus leaves, the PEF was used to determine the elastic modulus of the gel which was to be used as a substrate in the sample pad. The elastic modulus of a gel can be determined by placing the probe of the PEF on top of the gel and applying a voltage to the driving PZT layer using a function generator (Agilent 33220A) to exert a force on the gel, which in turn induces a piezoelectric voltage across the sensing PZT layer. The output was recorded using an oscilloscope (Agilent Technologies DSO3062A) and the elastic modulus, E, determined using the following mathematical equation:

$$E = ( 1 / 2 ) {( π / 2 )}^{1 / 2} ( 1 - {v}^{2} ) K ( Vin 0 - Vin ) / Vin ) \qquad Equation 1$$

where V_{in,0} and Vᵢₙ are the induced voltages without and with the tissue, respectively, vis Poisson's ratio of the tissue (which in this case is equal to 0.5 due to the incompressibility of water contained in the tissue), A is the contact area of the probe, and K is the effective spring constant of the PEF 19.

The PEF was lowered using an XYZ positioner until the probe touched the leaf (sample) surface, which was captured by a spike in induced voltage on the oscilloscope. The PEF was then lowered another 0.5 mm to ensure optimum contact between the sample and the 0.4 mm probe. For each sample, the PEF was excited with 2, 3, 4, 5, 6 and 7 volts and its respective induced voltage was obtained in triplicate.

Total genomic DNA was extracted from all leaves examined via the PEF using a modified Qiagen DNeasy Plant mini kit protocol (Qiagen; Germantown, Maryland, USA) as described by MM Doud et al. (2017, “Solar Thermotherapy Reduces the Titer of Candidatus Liberibacter Asiaticus and Enhances Canopy Growth by Altering Gene Expression Profiles in HLB-Affected Citrus Plants,” Hort. Res. 4, doi: ARTN 17054) using the midrib of each of the leaves. Assessment of the extracted DNA was performed using a DeNOVIX DS-11+ spectrophotometer (DeNOVIX; Wilmington, Delaware, USA). DNA was stored at −80° C. until assayed for the presence or absence of Las.

Assays for the presence/absence of Las were performed using the Applied Biosystems StepOnePlus Real-Time PCR System (ThermoFisher Scientific; Waltham, Massachusetts, USA) using the Las-specific 16S rDNA primer/probe set as described by LJ Zhou et al. (2011, “Diversity and Plasticity of the Intracellular Plant Pathogen and Insect Symbiont “Candidatus Liberibacter asiaticus,” Appl. Environ. Microbiol. 77:6663-6673). The primers used were HLBasf (having the sequence TCGAGCGCGTATGCGAATACG; set forth in SEQ ID NO: 1), BLBr (having the sequence GCGTTATCCCGTAGAAAAAGGTAG; set forth in SEQ ID NO: 2), and the probe used was HLBp (having the sequence AGACGGGTGAGTAACGCG; set forth in SEQ ID NO: 3). The probe HLBp was labeled with 6-carboxyfluorescein (FAM) at the 5′ end and lowa Black FQ at the 3′ end. TaqMan Fast Universal PCR Master Mix (2×), No AmpErase UNG (Life Technologies, Foster City, California, USA) was used in conjunction with the HLBasf/HLBr (5 nmol) primers and HLBp (2.5 nmol) probe, and 100 ng of total genomic DNA in a reaction volume totaling 15 μL. Amplification was performed in triplicate using the ‘fast’ temperature mode using cycling parameters described by MM Doud et al. (Supra). The presence of Las was defined as threshold cycle (Ct) values lower than 36. An internal control targeting the single-copy nuclear malate dehydrogenase (MDH) gene was used as described by ML Keremane et al. (2017, “An Improved Reference Gene for Detection of “Candidatus Liberibacter asiaticus” Associated with Citrus Huanglongbing by qPCR and Digital Droplet PCR Assays,” Plants 10, doi: ARTN 2111).

### Example 2

### PEF Modification to Accommodate Soft Leaf Tissue

Leaf tissue elastic modulus was determined by measuring changes in electrical voltage obtained with a modified piezoelectric device PEF and a sample pad.

As seen in FIG. 1, the design of the leaf elasticity tester used herein consisted of two piezoelectric layers sandwiching a stainless-steel layer in a cantilever structure with a thin rod-shaped metal probe located at the tip of the sensing piezoelectric layer for contacting the sample. A DC voltage was applied across the driving piezoelectric layer to apply a force at the tip of the cantilever so that the cantilever would bend. This generated an axial displacement and axial force at the tip, which was correlated with the induced voltage (Vin) across the sensing PZT generated by the bending of cantilever. Variations in the electrical voltage were measured using an oscilloscope.

When the rod-shaped probe at the cantilever tip was in contact with the sample, the induced voltage at the sensing PZT layer was reduced due to the reaction force by the sample. Induced voltages in the sensing PZT generated by the same applied voltage in the driving PZT were recorded as V_{in,0} when the probe was not touching the sample. The induced voltages obtained when the probe was in contact with the sample were recorded as Vᵢₙ. The elastic modulus, E, of the leaf tissue was then calculated using Equation #1, shown above. In practice, each PEF elasticity measurement required the application of multiple, discrete, applied voltages ranging between 2 and 7 V with a set of corresponding V_{in,0}. When the rod-shaped probe of the PEF was brought in contact with the sample surface, a set of Vᵢₙ, smaller than the corresponding V_{in,0} were generated by the same applied voltage. The elastic modulus was calculated for four different citrus species (Grapefruit, Pumelo, Lemon, and Valencia) and included both healthy and HLB-affected samples.

The results obtained when using the soft leaf tissue elasticity tester described herein to detect HLB by measuring electric voltage and determining the clastic modulus of grapefruit leaves at early and late stage of infection are shown in FIG. 6A to FIG. 6C. The elastic modulus (kPA) determined for grapefruit leaves at different stages of HLB infection are shown in FIG. 6A. The Y axis shows the clastic modulus in kPa; the X axis shows the types of leaves. Filled squares (▪) indicate data from healthy, Las⁻ (GFT-1G) leaves, horizontal arrows [symbol] indicate data from asymptomatic, Las⁺ (GFT-2G) leaves, upward facing triangles (▴) indicate data from Blotchy Mottle, Las⁺ (GFT-3G) leaves, and downward facing triangles (▾) indicate data from Yellow, Las⁺ (GFT-4G) leaves. FIG. 6B shows the normal distribution of elastic modulus values determined for Las-(GFT-1G) and Las⁺ (GFT-2G, GFT-3G, and GFT-4G) grapefruit leaves. The Y axis shows the leaf count; the X axis shows the elastic modulus in KPa. Solid bars represent Las-leaves, striped bars represent Las⁺ leaves. FIG. 6C shows the receiver operating characteristic (ROC) curve for healthy vs. diseased grapefruit leaves. Table 2 below shows the cutoff of 611 kPa, deduced from the graphs in FIG. 6A and FIG. 6B to achieve a sensitivity of 93% and a specificity of 89% between healthy and diseased grapefruit leaves. A dashed line in FIG. 6A and FIG. 6B shows the calculated grapefruit leaf elastic modulus cut-off. For GFT-1G, eleven different samples were tested in triplicate whereas for GFT-2G, GFT-3G and GFT-4G, six different samples were tested in triplicate with error bars representing the standard deviation of the determined elastic modulus.

TABLE 2
Grapefruit Data Summary

| Cutoff (kPa) | Sensitivity (%) | Specificity (%) |
|---|---|---|
| 611 | 93 | 89 |

The results obtained when using a soft leaf tissue elasticity tester described herein to detect HLB by measuring electric voltage with the soft leaf tissue elasticity tester described herein, and determining the elastic modulus of pummelo at early and late stages of infection are shown in FIG. 6A to FIG. 6C. The elastic modulus (kPa) determined for pummelo leaves at different stages of HLB infection are shown in FIG. 6A. The Y axis shows the elastic modulus in kPa; the X axis shows the leaf types. Filled squares (▪) indicate data from healthy PUM-1G (Las−) leaves, filled circles (●) indicate data from asymptomatic PUM-2G (Las+) leaves, and upward facing triangles indicate data from symptomatic PUM-3G (Las+) leaves. FIG. 6B shows the normal distribution of the elastic modulus values determined for Las− (PUM-1G) and Las+ (PUM-2G and PUM-3G) pummelo leaves. The Y axis shows the pummelo leaf count; the X axis shows the elastic modulus in KPa. Solid bars represent Las-pummelo leaves, striped bars represent Las⁺ pummelo leaves. Receiver operating characteristic (ROC) curve for healthy vs diseased pummelo leaves is shown in FIG. 6C. Table 3 below shows the cutoff of 598 kPa deduced from the graphs in FIG. 6A and FIG. 6B to achieve a 96% sensitivity and a 95% specificity between healthy and diseased pummelo leaves. A dashed line in FIG. 6A and FIG. 6B shows the calculated pumelo leaf elastic modulus cut-off. For PUM-1G, PUM-2G and PUM-3G six different samples were tested in triplicate with the error bars representing the standard deviation of the measurements.

TABLE 3
Pomelo Data Summary

| Cutoff (kPa) | Sensitivity (%) | Specificity (%) |
|---|---|---|
| 598 | 96 | 95 |

The results obtained when using the soft leaf tissue elasticity tester described herein to detect HLB by measuring electric voltage with the soft leaf tissue elasticity tester described herein, and determining the elastic modulus of lemon at early and late stages of infection are shown in FIG. 8A to FIG. 8C. The elastic modulus (kPa) determined for lemon leaves at different stages of HLB infection is shown in FIG. 8A. The Y axis shows the elastic modulus in kPa; the X axis shows the leaf types. Filled squares (▪) indicate data from healthy LEM-1G (Las−) leaves, filled circles (●) indicate data from asymptomatic LEM-2G (Las+) leaves, and upward-facing triangles (▴) indicate data from symptomatic LEM-3G (Las+) leaves. The normal distribution of the elastic modulus values determined for Las+ (LEM-1G) and Las-(LEM-2G and LEM-3G) lemon leaves is shown in FIG. 8B. The Y axis shows the lemon leaf count; the X axis shows the clastic modulus in KPa. Solid bars represent Las-lemon leaves, striped bars represent Las⁺ lemon leaves. The receiver operating characteristic (ROC) curve for healthy vs diseased lemon leaves is shown in FIG. 8C. Table 4 below shows the cutoff of 575 kPa deduced from the graphs in FIG. 8A and FIG. 8B to achieve a sensitivity of 89% and a specificity of 84% between healthy and diseased lemon leaves. A dashed line in FIG. 8A and FIG. 8B shows the calculated lemon leaf elastic modulus cut-off. For LEM-1G, LEM-2G and LEM-3G six different samples were tested in triplicate with error bars representing the standard deviation of the measurement.

TABLE 4
Lemon Data Summary

| Cutoff (kPa) | Sensitivity (%) | Specificity (%) |
|---|---|---|
| 598 | 89 | 84 |

The results obtained when using soft leaf tissue elasticity tester described herein to detect HLB by measuring electric voltage with the soft leaf tissue elasticity tester described herein, and determining the elastic modulus of Valencia sweet orange at early and late stages of infection are shown in FIG. 9A to FIG. 9C. The clastic modulus (kPa) determined for Valencia sweet orange leaves at different stages of HLB infection is shown in FIG. 9A. The Y axis shows the elastic modulus in kPa; the X axis shows the leaf types. Filled squares (▪) indicate data from healthy VAL-1G (Las−) leaves, filled circles (●) indicate data from asymptomatic VAL-2G (Las+) leaves, upward-facing triangles (▴) indicate data from symptomatic VAL-3G (Las+) leaves, and downward-facing triangles (▾) indicate data from yellow VAL-4G (Las+). The normal distribution of the elastic modulus values determined for Las−(VAL-1G) and Las+ (VAL-2G, VAL-3G and VAL-4G) Valencia leaves is shown in FIG. 9B. The Y axis shows the lemon leaf count; the X axis shows the elastic modulus in KPa. Solid bars represent Las-Valencia orange leaves, striped bars represent Las⁺ Valencia orange leaves. The receiver operating characteristic (ROC) curve for healthy vs diseased Valencia leaves is shown in FIG. 9C. Table 5 below shows the cutoff of 602 kPa deduced from the graphs in FIG. 9A and FIG. 9B to achieve a sensitivity of 95% and a specificity of 84% between healthy and diseased Valencia leaves. A dashed line in FIG. 9A and FIG. 9B shows the calculated Valencia sweet orange leaf elastic modulus cut-off. For VAL-1G, VAL-2G, VAL-3G and VAL-4G six different samples were tested in triplicate with error bars representing the standard deviation of the measurement.

TABLE 5
Valencia Data Summary

| Cutoff (kPa) | Sensitivity (%) | Specificity (%) |
|---|---|---|
| 598 | 95 | 84 |

This Example shows that results from four different types of citrus demonstrated a direct correlation between the elastic modulus and the presence of HLB.

We claim:

1. A soft leaf tissue elasticity tester comprising a piezoelectric finger (PEF) and a sample pad, and optionally comprising a DC voltage and a voltage detector in communication with the PEF.

2. The soft leaf tissue elasticity tester of claim 1, wherein the PEF comprises:
a first piezoelectric layer having a first length and being configured for application of a voltage thereto;
a second piezoelectric layer having a second length;
an optional non-piezoelectric layer, a proximal portion of which is located between the first piezoelectric layer and the second piezoelectric layer; and
a probe having a first end opposite a second end, the first end coupled at a distal end of the first piezoelectric layer or the non-piezoelectric layer when present, and the second end having a contact area.

3. The soft leaf tissue elasticity tester of claim 2, wherein the first piezoelectric layer, the second piezoelectric layer, and the non-piezoelectric layer when present, are coupled at a distal end from the probe.

4. The soft leaf tissue elasticity tester of claim 2, wherein the non-piezoelectric layer is present.

5. The soft leaf tissue elasticity tester of claim 4, wherein the first piezoelectric layer, the second piezoelectric layer, and the non-piezoelectric layer are coupled at a distal end from the probe.

6. The soft leaf tissue elasticity tester of claim 2, wherein the probe width is from about one hundred times smaller than the soft leaf tissue sample thickness to as large as the sum of the thickness of the soft tissue sample and that of the sample pad.

7. The soft leaf tissue elasticity tester of claim 2, wherein the sample pad has a depth larger than at least about two times the width of the probe.

8. The soft leaf tissue elasticity tester of claim 2, wherein the sample pad is a gel with an elastic modulus smaller than a calculated elastic modulus of a soft leaf tissue to be measured.

9. The soft leaf tissue elasticity tester of claim 1, wherein a proximal portion of the sample pad is coupled to a proximal portion of the PEF.

10. The soft elasticity tester of claim 2, wherein a proximal portion of the first piezoelectric layer, the second piezoelectric layer, and the non-piezoelectric layer when present are coupled to a proximal portion of the sample pad.

11. The soft elasticity tester of claim 2, wherein the non-piezoelectric layer is present; and a proximal portion of the first piezoelectric layer, the second piezoelectric layer, and the non-piezoelectric layer are coupled to a proximal portion of the sample pad.

12. The soft leaf tissue elasticity tester of claim 10, wherein the soft leaf tissue elasticity tester is automated.

13. A method for screening a plant for disease, the method comprising:
determining an elastic modulus of a soft leaf tissue from the plant;
comparing the elastic modulus determined for the soft leaf tissue from the plant to a calculated elastic modulus cut-off; and
concluding that the plant is diseased if the elastic modulus determined for the soft leaf tissue from the plant is larger than the calculated elastic modulus cut-off.

14. The method of claim 13, wherein the elastic modulus cutoff is calculated to achieve at least about 80% sensitivity and at least about 80% specificity between healthy and diseased plant leaf tissue.

15. The method of claim 13, wherein the elastic modulus is determined using measurements obtained with the soft leaf tissue elasticity tester comprising a PEF and a sample pad, and optionally comprising a DC voltage and a voltage detector in communication with the PEF.

16. The method of claim 13, wherein the elastic modulus is determined using measurements obtained with the soft leaf tissue elasticity tester comprising a PEF and a sample pad, and optionally comprising a DC voltage and a voltage detector in communication with the PEF; wherein a proximal portion of the sample pad is coupled to a proximal portion of the PEF.

17. The method of claim 13, wherein the elastic modulus is determined using measurements obtained with a soft leaf tissue elasticity tester comprising a PEF and a sample pad; and optionally comprising a DC voltage and a voltage detector in communication with the PEF; wherein the PEF comprises:
a first piezoelectric layer having a first length and being configured for application of a voltage thereto;
a second piezoelectric layer having a second length;
an optional non-piezoelectric layer, a proximal portion of which is located between the first piezoelectric layer and the second piezoelectric layer; and
a probe having a first end opposite a second end, the first end coupled at a distal end of the first piezoelectric layer or the non-piezoelectric layer when present, and the second end having a contact area.

18. The method of claim 13, wherein the disease is a viral infection, a bacterial infection, or a fungal infection.

19. The method of claim 18, wherein the disease is a bacterial infection, and the bacterial infection is Huanglongbing (HLB).

20. The method of claim 13, wherein the screened plant is a member of the Rutaceae family.

21. The soft leaf tissue elasticity tester of claim 1, wherein the PEF consists of a driving piezoelectric layer and a sensing piezoelectric layer sandwiching a stainless-steel layer in a cantilever structure with a thin rod-shaped metal probe having a first end opposite a second end, the first end coupled at a distal end of the sensing piezoelectric layer for contacting the sample.


SEQUENCE LISTING (3 sequences)

SEQ ID NO: 1 - DNA, 21 bp, synthetic construct
  source 1..21: mol_type = other DNA
tcgagcgcgtatgcgaatacg

SEQ ID NO: 2 - DNA, 24 bp, synthetic construct
  source 1..24: mol_type = other DNA
gcgttatcccgtagaaaaaggtag

SEQ ID NO: 3 - DNA, 18 bp, synthetic construct
  source 1..18: mol_type = other DNA
agacgggtgagtaacgcg